(19) United States
(12) Patent Application Publication
(10) Pub. No.: US 2008/0306610 A1
(43) Pub. Date: Dec. 11, 2008

(54) TISSUE PROCESSING FOR NONIMMUNOGENIC IMPLANTS

(75) Inventors: Hali Wang, The Hills, TX (US); Hui Liu, Austin, TX (US); Hai-Qing Xian, Austin, TX (US); Jian Q. Yao, Austin, TX (US); Victor Zaporojan, Austin, TX (US)

Correspondence Address: WOOD, HERRON & EVANS, LLP (ZIMMER), 2700 CAREW TOWER, 441 VINE STREET, CINCINNATI, OH, 45202, US

(73) Assignee: ZIMMER ORTHOBIOLOGICS, INC., Austin, TX (US)

(21) Appl. No.: 11/759,679

(22) Filed: Jun. 7, 2007

(51) Int. Cl. A61F 2/02; C12N 5/02

(52) U.S. Cl. 623/23.72; 435/378; 435/381

(57) ABSTRACT

Methods for processing tissues to render them suitable for implantation, e.g. in an orthopedic site. Tissues are rendered substantially acellular and substantially nonimmunogenic by exposure to processes that result in cell lysis, increasing permeability of the extracellular matrix, degrading the debris from lysis, and removing the debris. Methods of forming tissue implants, kits for processing tissue implants, and methods of using tissue implants are also disclosed.

### TECHNICAL FIELD

Preparation of nonimmunogenic implants from tissue by treatments that devitalize cells, extract cellular debris, and reduce immunoantigens.

### BACKGROUND

Implants of tissue, obtained from various sources, may be used to repair tissue defects. Obtaining such tissue may be problematic. For example, autografts may require two surgical procedures on the patient being treated with the implant: one to remove the tissue from one site; and the other to implant the tissue at another site. Autografts also may require processing prior to implantation, causing a delay in treatment. Allografts may be supply limited or expensive to procure, and the tissue must be processed to be rendered noninfectious and as nonimmunogenic as possible. Xenografts are the most abundant and least costly tissue sources, but the tissue must also be processed to be rendered noninfectious and as nonimmunogenic as possible.

The ease and success of tissue processing may depend upon the method used and the tissue type, among other factors. As one example, processing is more difficult with connective tissue, such as cartilage or meniscus, because some connective tissue does not readily permit penetration by processing reagents.

Other methods are thus desirable.

### SUMMARY OF THE INVENTION

One embodiment is a processing method for tissue that renders the tissue substantially acellular and substantially nonimmunogenic, and thus suitable for implantation. A tissue specimen including cells and extracellular matrix (ECM) is treated by the following steps: (i) lysing at least one cell, which results in production of cellular debris, (ii) enhancing the permeability of the ECM, (iii) degrading the cellular debris, and (iv) removing the degraded cellular debris. Any of the treatment steps may be repeated. The resulting processed tissue is substantially acellular and substantially nonimmunogenic, and may be implanted in a human or non-human mammal, either immediately after processing or after a storage period.

Another embodiment is a kit for preparing such a tissue specimen. The kit includes at least one detergent or instructions for preparing at least one detergent, at least one nuclease or instructions for preparing at least one nuclease, at least one hydrolase or instructions for preparing at least one hydrolase, and instructions for treating a tissue specimen with the at least one detergent, nuclease or hydrolase to result in a substantially nonimmunogenic and substantially acellular tissue specimen capable of implantation. The kit may additionally contain at least one salt or instructions for preparing a salt, at least one buffer or instructions for preparing a buffer, at least three different alcohol concentrations or instructions for preparing three different alcohol concentrations, and a wash solution or instructions for preparing a wash solution.

Another embodiment is a method of implanting an orthopedic tissue graft. The tissue to be implanted is treated by lysing at least one cell resulting in production of cellular debris, enhancing a permeability of the extracellular matrix of the tissue graft, degrading the cellular debris, and removing the degraded cellular debris. Any of the treatments may be repeated, and the resulting tissue specimen is substantially acellular and substantially nonimmunogenic. These and other embodiments will be further appreciated with respect to the following figures, detailed description, and examples.

### BRIEF DESCRIPTION OF THE DRAWINGS

This application contains at least one drawing executed in color. A Petition under 37 C.F.R. §1.84 requesting acceptance of the color drawings is filed separately on even date herewith. Copies of this patent or patent application publication with color drawing(s) will be provided by the Office upon request and payment of the necessary fee.

FIG. 1 shows a generalized flowchart of one embodiment of the method.

FIG. 2 shows a detailed flowchart of one embodiment of the method.

FIG. 3A shows histochemical staining with Hoechst stain of untreated porcine menisci.

FIG. 3B shows histochemical staining with phalloidin stain of untreated porcine menisci.

FIG. 4A shows histochemical staining with Hoechst stain of porcine menisci processed using one embodiment of the method.

FIG. 4B shows histochemical staining with phalloidin stain of porcine menisci processed using one embodiment of the method.

FIG. 5A shows histochemical staining with Hoechst stain of porcine menisci processed using another embodiment of the method.

FIG. 5B shows histochemical staining with phalloidin stain of porcine menisci processed using another embodiment of the method.

FIG. 6A shows histochemical staining with Hoechst stain of porcine menisci processed using another embodiment of the method.

FIG. 6B shows histochemical staining with phalloidin stain of porcine menisci processed using another embodiment of the method.

FIG. 7A shows histochemical staining with Hoechst stain of porcine menisci processed using another embodiment of the method.

FIG. 7B shows histochemical staining with phalloidin stain of porcine menisci processed using another embodiment of the method.

### DETAILED DESCRIPTION

Tissue processing methods that devitalize cells in a tissue specimen, extract cellular debris, and reduce immunoantigens in the extracellular components to result in substantially acellular and substantially nonimmunogenic tissue capable of implantation in a human or non-human mammal is provided.

In one embodiment, a tissue specimen containing cells and extracellular matrix (ECM) is obtained by excising tissue from a body site using methods known to one skilled in the art. In some embodiments, the tissue specimen is, or is derived from, a tissue such as cartilage, meniscus, ligament, tendon, nucleus pulposus, annulus fibrosus, skin, bone, and/or blood vessels. Autograft, allograft, and/or xenograft tissue may be used.

Tissue is processed to be rendered substantially acellular and substantially nonimmunogenic. Processing is by lysing at least one cell resulting in production of cellular debris, enhancing permeability of the ECM, degrading the cellular debris, and removing the degraded cellular debris. Any or all of the treatments may be repeated for additional processing. For example, connective tissue such as meniscus may need more steps to increase its permeability. In one embodiment, cell lysis occurs before enhancing ECM permeability. In other embodiments, cell lysis may occur after or both before and after enhancing ECM permeability. Cell lysis (devitalization) results in cellular debris, and removal of this debris results in decellularization. Removal of the cellular debris also decreases immunogenicity of the tissue. Denaturing and/or degrading ECM proteins further decrease immunogenicity of the tissue and also increase permeability of the ECM to increase reagent penetration. Increased reagent penetration facilitates more complete cell lysis and cell debris removal.

In various embodiments, cells are lysed by chemical treatment, physical treatment, or both chemical and physical treatments. Cell lysis by physical treatment includes, for example, exposing the tissue to freezing and thawing. In some embodiments, this may be repeated in a number of freeze/thaw cycles. Cell lysis by physical treatment also includes high pressure and/or vacuum, sonication, and/or single or multiple cycles of mechanical compression. Cell lysis by chemical treatment includes, for example, exposing the tissue to a buffer solution having an osmolality that is lower than physiological (i.e., a hypotonic buffer). Osmolality is expressed as milliosmoles per kilogram of water (mOsm/kg water) and generally physiological osmolality is about 290 mOsm/kg water. In one embodiment, the hypotonic buffer has an osmolality in the range of about 180 mOsm/kg water to about 220 mOsm/kg water. The hypotonic buffer may contain various salt and/or buffer components such as Tris hydroxymethylaminoethane (Tris), phosphate-buffered saline (PBS), potassium chloride, and/or sodium chloride. Cell lysis by chemical treatment includes enzyme digestion using, for example, lysozyme. Cell lysis by chemical treatment includes exposing the tissue to reagents that solubilize lipids and/or cellular membranes. Such reagents include detergents, for example, Triton® X-100 and sodium dodecyl sulfate (SDS), and/or organic solvents, for example, phenol and chloroform.

In embodiments, cell lysis occurs in the presence of at least one protease inhibitor to inactivate the endogenous enzymes. Protease inhibitors are known to one skilled in the art and include, but are not limited to, phenylmethylsulfonylfluoride (PMSF) and ethylenediaminetetraacetic acid (EDTA).

As described above, increasing the permeability of the ECM allows increased reagent penetration into the tissue. In various embodiments, the permeability of the ECM may be increased using one or more agents such as a hypertonic buffer, a detergent, and/or an enzyme. For example, when the tissue is derived from connective tissue, such as meniscus, enzyme(s) are used to increase ECM permeability.

In embodiments where a detergent is used to increase ECM permeability, the detergent may be, but is not limited to, octylphenol ethoxylate (Triton X-100), polyoxyethylene sorbitan monolaurate (Tween-20), octylphenolpoly (ethyleneglycolether) (NP-40), sodium dodecyl sulfate (SDS), and/or sodium deoxycholate (SDC). In one embodiment, octylphenol ethoxylate is present at a concentration ranging from about 0.01% w/v to about 10% w/v. In another embodiment, octylphenol ethoxylated is present at a concentration ranging from about 0.1% w/v to about 10% w/v. In another embodiment, octylphenol ethoxylated is present at a concentration ranging from about 0.1% w/v to about 3% w/v. In various embodiments, the detergent concentration is in the range from about 0.01% w/v to about 10% W/v.

In embodiments where an enzyme is used to increase the ECM permeability, enzymes may be, but are not limited to, hydrolases. Non-limiting examples of hydrolases include hyaluronidase, chondroitinase ABC, collagenase, trypsin, and/or lipase. In one embodiment, hyaluronidase is present at a concentration ranging from about 0.1 mg/ml to about 30 mg/ml with activity ranging from about 400 U/mg to about 1000 U/mg. In another embodiment, hyaluronidase is present at a concentration ranging from about 1 mg/ml to about 10 mg/ml with activity ranging from about 400 U/mg to about 1000 U/mg. In various embodiments, the tissue is exposed to enzyme at a temperature ranging from about 4° C. to about 40° C. In various embodiments, the concentration of trypsin is in the range of about 0.01% w/v to about 1 % w/v, the concentration of chondroitinase ABC and collagenase is in a range from about 0.01 U/ml to about 2 U/ml, and the concentration of lipase is in the range of about 50 U/ml to about 1,000 U/ml, where each concentration is independent and relates to the total composition.

In one embodiment, cellular debris that results from lysing, with or without increasing ECM permeability, is degraded and removed. In one embodiment, degradation occurs by exposing the tissue to enzyme (e.g., nuclease, protease, lipase, etc.), detergent, organic solvent, and/or a protein-denaturing agent.

In embodiments using a nuclease, the nuclease may be a deoxyribonuclease or a ribonuclease. In one embodiment, the nuclease is present at a concentration ranging from about 1 U/ml to about 1000 U/ml. In another embodiment, the nuclease is present at a concentration ranging from about 20 U/ml to about 200 U/ml, where the concentration is independent of other reagents and relates to the total composition. In various embodiments, the tissue is exposed to the nuclease at a temperature ranging from about 4° C. to about 40° C.

In embodiments using a protease, the protease may be, for example, trypsin and/or α-chymotrypsin. In one embodiment, the protease is present at a concentration ranging from about 0.01% w/v to about 1% w/v. In another embodiment, the protease is present at a concentration ranging from about 0.1% w/v to about 0.5% w/v. In various embodiments, the tissue is exposed to the protease at a temperature ranging from about 4° C. to about 40° C. In another embodiment, an esterase can be used at a concentration in the range of about 1 U/ml to about 100 U/ml, where the concentration is independent of other reagents and relates to the total composition.

In embodiments using a detergent or organic solvent, the detergent or organic solvent may be sodium dodecyl sulfate (SDS), sodium deoxycholate (SDC), octylphenol ethoxylated (Triton X-100), polyoxyethylene sorbitan monolaurate (Tween-20), octylphenolpoly(ethyleneglycolether) (NP-40), and/or tributyl phosphate (TnBP). In one embodiment, the detergent is present at a concentration ranging from about 0.01% w/v to about 10% w/v, where the concentration is independent of other reagents and relates to the total composition. In another embodiment, the detergent is present at a concentration ranging from about 0.1% w/v to about 10% w/v. In another embodiment, the detergent is present at a concentration ranging from about 0.1% w/v to about 3% w/v. In one embodiment, the organic solvent is present at a concentration ranging from about 0.1% v/v to about 100% v/v.

In embodiments using a protein-denaturing agent, the protein-denaturing agent may be urea or guanidine at a concentration in the range of about 0.01 M to about 8 M for urea and about 0.01 M to about 6 M for guanidine, where the concentration is independent of other reagents and relates to the total composition. Other examples of protein-denaturing agents that may be used include surface-active agents such as detergents, including SDS and Triton® X-100, and/or reducing agents, including isopropanol, formaldehyde, and formic acid.

Following one or more treatments to degrade cellular debris, tissue is washed to remove the debris. In various embodiments, washing may be with water, an aqueous solution such as a saline solution, or an aqueous buffer such as a Tris buffer, a phosphate buffer, or a Hank's balanced salt solution (HBSS). In one embodiment, tissue is washed by incubating in a solution and subjecting it to agitation, for example, on an orbital mixer, shaker, or tilt table. In other embodiments, washing may occur under high or low pressure and/or coupled with sonication, to enhance the penetration and/or removal of cellular debris and treatment solutions. Any extent of washing may be performed to result in removing a substantial portion of the debris. In certain embodiments, tissue is washed for a time from about one hour to about 24 hours.

In other embodiments, the tissue may also be exposed to a high salt (HS), or a high salt and high sugar (HS-HS), solution. In one embodiment, NaCl is the salt and sucrose is the sugar. In one embodiment, NaCl is present at a final concentration ranging from about 11% w/v to about 29% w/v. In one embodiment, sucrose is present at a final concentration ranging from about 30% w/v to about 80% w/v. In one embodiment, NaCl is present at a final concentration of about 29% w/v and sucrose is present at a final concentration ranging from about 61% w/v to about 63% w/v, where the concentration is independent of other reagents and relates to the total composition. In one embodiment, the osmolality of the HS-HS solution is greater than 4500 milliosmoles and up to and including the saturation point of the solvent. In another embodiment, the osmolality of the HS-HS solution is greater than 6000 milliosmoles and up to and including the saturation point of the solvent. In another embodiment, the osmolality of the HS-HS solution ranges from about 8200 milliosmoles to about 8900 milliosmoles. In one embodiment, the HS-HS solution has a refractive Index ranging from about 45% to about 70%. In another embodiment, the HS-HS solution has a refractive index ranging from about 60% to about 65%.

In other embodiments, the tissue may be dehydrated and optionally rehydrated at any step during processing. In one embodiment, dehydration and rehydration occurs by exposing the tissue to sequentially increasing concentrations of alcohol, either the same or different alcohol, followed by sequentially decreasing concentrations of alcohol, either the same or different alcohols. For example, tissue is exposed to an alcohol at a first concentration, then to an alcohol at a second concentration higher than the first concentration to achieve a desired level of tissue dehydration. If desired, the dehydrated tissue is further exposed to an alcohol at a third concentration lower than the second concentration, to achieve a desired level of rehydration. Any number of alcohol concentrations may be used. In one embodiment, the increasing alcohol concentrations start at about 10% v/v alcohol up to 100% v/v alcohol. For example, increasing alcohol concentrations may be about 50% v/v and up to, about 55% v/v, about 60% v/v, about 65% v/v, about 70% v/v, about 75% v/v, about 80% v/v, about 85% v/v, about 90% v/v, about 95% v/v, to 100% v/v alcohol. In another embodiment, the decreasing alcohol concentrations start at 100% v/v alcohol down to about 10% v/v alcohol. For example, decreasing alcohol concentrations may be about 95% v/v and down to, about 90% v/v, about 85% v/v, about 80% v/v, about 75% v/v, about 70% v/v, about 65% v/v, about 60% v/v, about 55% v/v, to about 50% v/v alcohol. In one embodiment, the alcohol used at at least one concentration is ethanol. In one embodiment, the alcohol used at all concentrations is ethanol. In some embodiments, agitation is provided during the processing steps.

In any or all of the method steps, at least one antimicrobial agent may be included to decrease tissue bio-burden. For example, antibiotics such as penicillin, neomycin, streptomycin, etc. may be included with the reagents at any or all method steps.

In one embodiment, the reagents, or instructions for preparing reagents, and instructions for tissue processing are included in a kit. In one embodiment, the kit includes at least one detergent or instructions for preparing a detergent, at least one nuclease or instructions for preparing a nuclease, at least one hydrolase or instructions for preparing a hydrolase, and instructions for treating a tissue specimen with these reagents to result in a substantially nonimmunogenic and substantially acellular tissue. The kit may contain additional reagents, such as at least one salt or instructions for preparing a salt, at least one buffer or instructions for preparing a buffer, at least three different alcohol concentrations or instructions for preparing three different alcohol concentrations, and/or a wash solution or instructions for preparing a wash solution.

In one embodiment, the kit provides one or more detergents, such as octylphenol ethoxylate (Triton X-100), polyoxyethylene sorbitan monolaurate (Tween-20), octylphenolpoly(ethyleneglycolether) (NP-40), sodium dodecyl sulfate (SDS), and/or sodium deoxycholate (SDC), or instructions for preparing the detergent. In one embodiment, the kit provides one or more enzymes, such as hyaluronidase, chondroitinase ABC, collagenase, trypsin and/or lipase, or instructions for preparing a desired concentration of the enzyme(s).

The processed tissue prepared according to the above methods and rendered substantially acellular and substantially nonimmunogenic results in a tissue graft. In one embodiment, the tissue graft is implanted in an orthopedic tissue. For example, meniscus tissue subjected to the disclosed method showed a similar immune response as high molecular weigh polyethylene, used as a negative control because of its low antigenicity.

The following example further illustrates embodiments of the method.

### EXAMPLE

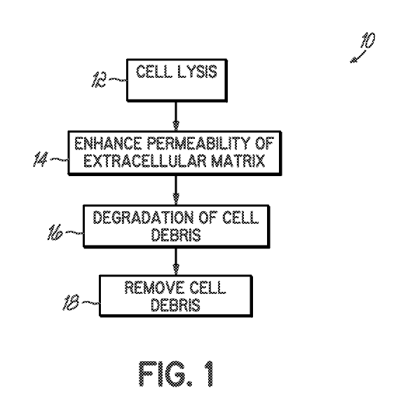

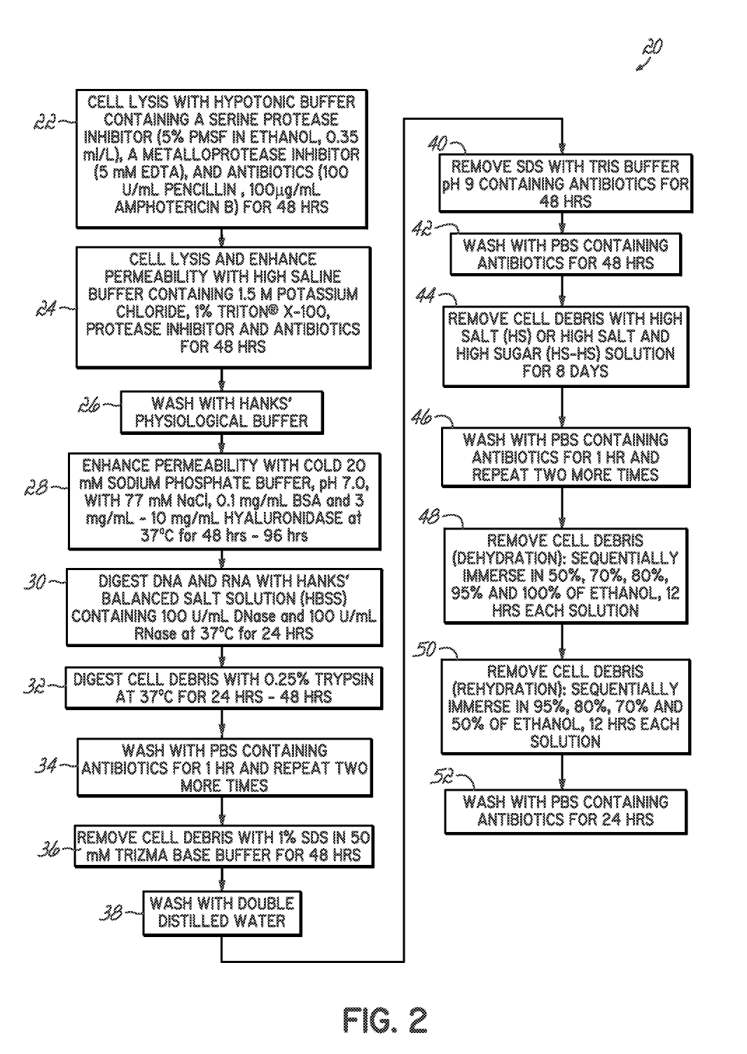

The general method steps (10) used in the Example are shown in FIG. 1. The tissue sample was treated to lyse cells (12), enhance permeability of extracellular matrix (14), degrade cell debris (16), and remove cell debris (18). A detailed flowchart (20) of the Example is shown in FIG. 2. Menisci obtained by dissection from 6-8 months old wild type pig hind limb were processed as described below. Unless otherwise specified, all steps were conducted at room temperature (about 20° C. to about 25° C.) with agitation at 120 rpm on an orbital mixer. Tissues were immersed in solutions at a concentration in the range from about 150 mg/ml to about 200 mg/ml.

Menisci (3.5 cm in length and 2.5 cm in width) were immersed in hypotonic 10 mM Tris buffer containing final concentrations of 0.35 ml/L phenylmethylsulfonylfluoride (PMSF) made from 5% PMSF in ethanol, 5 mM EDTA, 5 ml/L metalloprotease inhibitor, 100 U/ml penicillin/100 μg/ml streptomycin/0.25 μg/ml amphotericin B for 48 hours (22).

Menisci were then immersed in high saline buffer containing 1.5 M potassium chloride, 1% w/v Triton X-100, protease inhibitor (PMEF) and penicillin/streptomycin/amphotericin B for 48 hrs (24). Menisci were then rinsed with Hanks balanced salt solution (26) and immersed in cold 20 mM sodium phosphate buffer, pH 7.0, with 77 mM NaCl, 0.1 mg/ml BSA and 3 mg/ml hyaluronidase at 37° C. for 48 hours (28).

Menisci were then immersed in Hanks balanced salt solution containing 100 U/ml DNase and 100 U/ml RNase at 37° C. for 24 hours (30).

Menisci were then immersed in 0.25% trypsin at 37° C. for 48 hours (32) and then washed with PBS containing penicillin/streptomycin/amphotericin B for one hour; this step was repeated twice (34).

Menisci were then immersed in 1% w/v SDS in 50 mM Tris base buffer for 48 hours (36), and then rinsed with double distilled water (38) and immersed in Tris buffer pH 9 containing penicillin/streptomycin/amphotericin B for 48 hours (40).

Menisci were then washed with PBS containing penicillin/streptomycin/amphotericin B for 48 hours (42) and then immersed in high salt (HS) or high salt and high sugar (HS-HS) (the osmolality ranged from about 8200 milliosmoles to about 8900 milliosmoles and a refractive Index of about 63 brix %) solution for eight days (44). Menisci were then washed with PBS containing penicillin/streptomycin/amphotericin for one hour; this step was repeated twice (46).

Menisci were then dehydrated by sequentially immersing separately for 12 hours each in ethanol at the following concentrations: 50%, 70%, 80%, 95% and 100% to remove cell debris (48). Menisci were then rehydrated by sequentially immersing separately for 12 hours each in ethanol at the following concentrations: 95%, 80%, 70% and 50% ethanol solution to remove cell debris (50). Menisci were then washed with PBS containing penicillin/streptomycin/amphotericin B for 24 hrs (52).

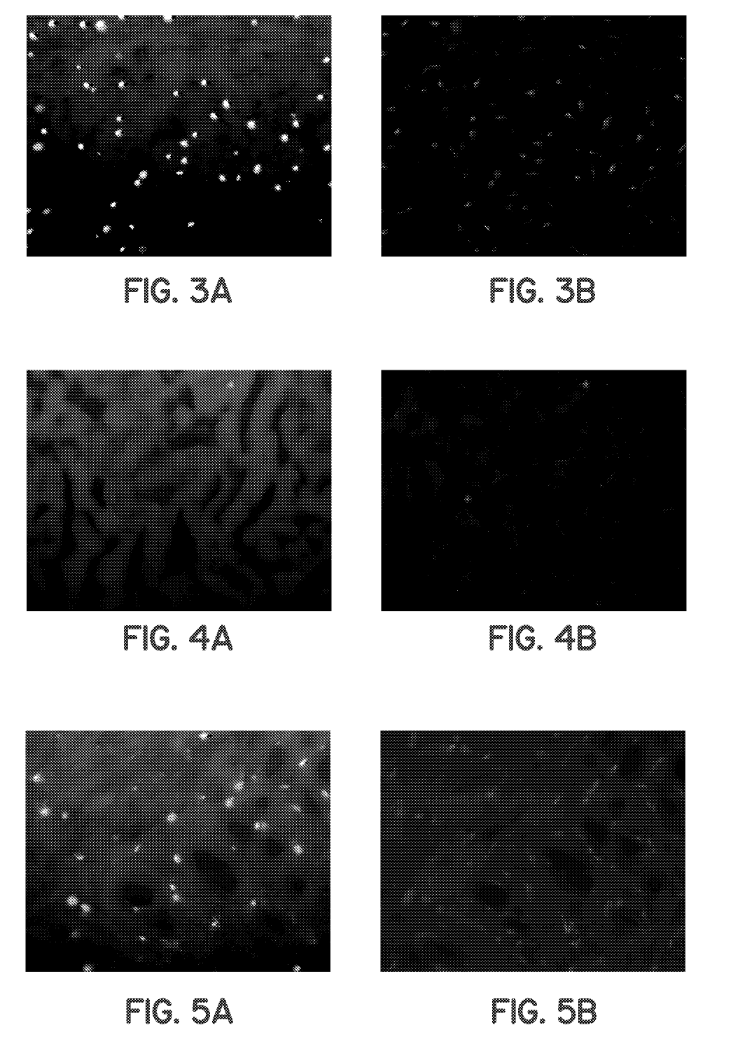

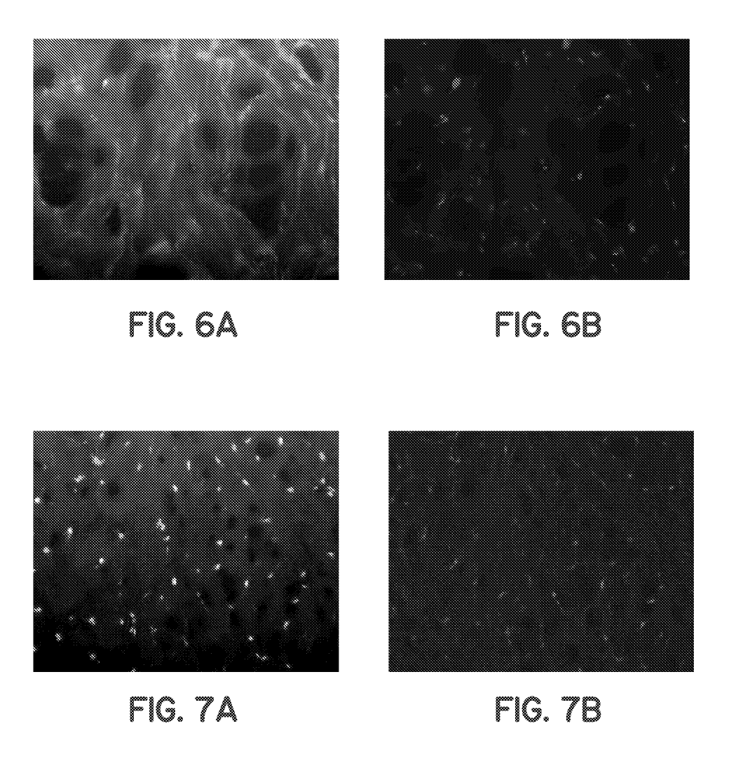

Histological sections of tissue unprocessed (control) and processed as described were stained with Hoechst (Invitrogen) to stain nuclei. (FIGS. 3A, 4A, 5A, 6A, and 7A), and with phalloidin (Invitrogen) to stain cytoskeletal protein actin (FIGS. 3B, 4B, 5B, 6B, and 7B). FIGS. 3A and 3B show unprocessed menisci. FIGS. 4A and 4B show processed menisci. Results of menisci processed as described, except for exclusion of the hyaluronidase and trypsin treatment, are shown in FIGS. 5A and 5B. Results of menisci processed as described, except for exclusion of Triton® X-100 and SDS, are shown in FIGS. 6A and 6B. Results of menisci processed as described, except for exclusion of detergents and trypsin, are shown in FIGS. 7A and 7B. The results showed that the complete decellularization process of the method (including lysing cells, increasing ECM permeability, degrading cellular debris, and removing degraded cellular debris) successfully devitalized wild type porcine menisci while omission of various steps resulted in a decrease in tissue decellularization.

It should be understood that the embodiments and examples described are only illustrative and are not limiting in any way. Therefore, various changes, modifications or alterations to these embodiments may be made or resorted to without departing from the spirit of the invention and the scope of the following claims.

What is claimed is:

1. A tissue specimen preparation method comprising:
treating a tissue specimen comprising cells and extracellular matrix (ECM), wherein the ECM possesses a permeability by:
lysing at least one cell resulting in production of cellular debris,
enhancing the permeability of the ECM,
degrading the cellular debris, and
removing the degraded cellular debris,
wherein any of the treatments may be repeated, and wherein the product is substantially acellular and substantially nonimmunogenic.

2. The method of claim 1 wherein lysing occurs after enhancing ECM permeability.

3. The method of claim 1 wherein the tissue specimen comprises cartilage, meniscus, ligament, tendon, skin, and blood vessels.

4. The method of claim 1 wherein lysing comprises exposing the tissue specimen to at least one of a chemical or a physical treatment.

5. The method of claim 4 wherein the physical treatment comprises freezing and thawing the tissue specimen.

6. The method of claim 4 wherein the chemical treatment comprises treating the tissue specimen with a hypotonic buffer solution.

7. The method of claim 6 wherein the hypotonic buffer solution has an osmolality that is lower than physiological, and comprises at least one of Tris hydroxymethylaminoethane (Tris), phosphate-buffered saline (PBS), potassium chloride or sodium chloride.

8. The method of claim 4 wherein the lysing further comprises exposing the tissue specimen to a protease inhibitor.

9. The method of claim 8 wherein the protease inhibitor is at least one of phenylmethylsulfonylfluoride (PMSF) or ethylenediaminetetraacetic acid (EDTA).

10. The method of claim 1 wherein enhancing ECM permeability comprises exposing the tissue specimen to at least one of a hypertonic buffer, a detergent, or an enzyme.

11. The method of claim 10 wherein the detergent comprises octylphenol ethoxylate (Triton X-100), polyoxyethylene sorbitan monolaurate (Tween-20), octylphenolpoly (ethyleneglycolether) (NP-40), sodium dodecyl sulfate (SDS), or sodium deoxycholate (SDC), or combinations thereof, and wherein the detergent is present at a concentration ranging from about 0.01% w/v to about 10% w/v.

12. The method of claim 11 wherein the detergent is present at a concentration ranging from about 0.1% w/v to about 3% w/v.

13. The method of claim 11 wherein octylphenol ethoxylated is present at a concentration ranging from about 0.1% w/v to about 10% w/v.

14. The method of claim 10 wherein the enzyme is at least one of hyaluronidase, chondroitinase ABC, collagenase, trypsin or lipase.

15. The method of claim 14 wherein hyaluronidase is present at a concentration ranging from about 0.1 mg/ml to about 30 mg/ml with activity ranging from about 400 U/mg to about 1000 U/mg.

16. The method of claim 14 wherein hyaluronidase is present at a concentration ranging from about 1 mg/ml to about 10 mg/ml with activity ranging from about 400 U/mg to about 1000 U/mg.

17. The method of claim 14 wherein trypsin is present at a concentration ranging from about 0.01% w/v to about 1% w/v.

18. The method of claim 14 wherein chondroitinase ABC is present at a concentration ranging from about 0.01 U/ml to about 2 U/ml.

19. The method of claim 14 wherein collagenase is present at a concentration ranging from about 0.01 U/ml to about 2 U/ml.

20. The method of claim 14 wherein lipase is present at a concentration ranging from about 50 U/ml to about 1,000 U/ml.

21. The method of claim 14 wherein the tissue specimen is exposed to enzyme at a temperature ranging from about 4° C. to about 40° C.

22. The method of claim 1 wherein degrading the cellular debris comprises exposing the tissue specimen to at least one of a nuclease, a protease, a lipase, a detergent, an organic solvent or a protein denaturing agent, or combinations thereof.

23. The method of claim 22 wherein the nuclease is at least one of deoxyribonuclease or ribonuclease.

24. The method of claim 22 wherein the nuclease is present at a concentration of about 1 U/ml to about 1000 U/mI.

25. The method of claim 22 wherein the nuclease is present at a concentration of about 20 U/ml to about 200 U/ml.

26. The method of claim 22 wherein the tissue specimen is exposed to the nuclease at a temperature ranging from about 4° C. to about 40° C.

27. The method of claim 22 wherein the protease is at least one of trypsin or α-chymotrypsin.

28. The method of claim 22 wherein the protease is present at a concentration ranging from about 0.01% w/v to about 1% w/v.

29. The method of claim 22 wherein the protease is present at a concentration ranging from about 0.1% w/v to about 0.5% w/v.

30. The method of claim 22 wherein the tissue specimen is exposed to the protease at a temperature ranging from about 4° C. to about 40° C.

31. The method of claim 22 wherein the detergent or organic solvent is at least one of sodium dodecyl sulfate (SDS), sodium deoxycholate (SDC), octylphenol ethoxylated (Triton X-100), polyoxyethylene sorbitan monolaurate (Tween-20), octylphenolpoly(ethyleneglycolether) (NP-40), or tributyl phosphate (TnBP), or combinations thereof.

32. The method of claim 22 wherein the detergent is present at a concentration ranging from about 0.1% w/v to about 10% w/v; the organic solvent from about 0.1% v/v to about 100% v/v.

33. The method of claim 22 wherein the detergent is present at a concentration ranging from about 0.5% w/v to about 5% w/v.

34. The method of claim 22 wherein the protein-denaturing agent is at least one of urea or guanidine.

35. The method of claim 1 wherein removing the cellular debris comprises washing the tissue specimen.

36. The method of claim 35 wherein the washing is with at least one of water, an aqueous solution, or an aqueous buffer.

37. The method of claim 36 wherein the aqueous buffer is at least one of a Tris buffer, a phosphate buffer or a Hank's balanced salt solution (HBSS).

38. The method of claim 35 further comprising exposing the tissue specimen to a high salt (HS) or a high salt and high sugar (HS-HS) solution.

39. The method of claim 35 further comprising dehydrating and rehydrating the tissue specimen.

40. The method of claim 39 wherein dehydrating and rehydrating comprises exposing the tissue specimen to a series of increasing alcohol concentrations followed by a series of decreasing alcohol concentrations such that dehydrating comprises exposing the tissue specimen to an alcohol at a first concentration, then to an alcohol at a second concentration higher than the first concentration, and rehydrating comprises exposing the dehydrated tissue specimen to an alcohol at a third concentration lower than the second concentration.

41. The method of claim 40 wherein the increasing alcohol concentrations start at about 10% v/v alcohol up to 100% v/v alcohol.

42. The method of claims 40 wherein the decreasing alcohol concentrations start at 100% v/v alcohol down to about 10% v/v alcohol.

43. The method of claim 40 wherein the increasing alcohol concentrations are selected from at least two of the following: about 50% v/v, about 55% v/v, about 60% v/v, about 65% v/v, about 70% v/v, about 75% v/v, about 80% v/v, about 85% v/v, about 90% v/v, about 95% v/v, and about 100% alcohol.

44. The method of claims 40 wherein the decreasing alcohol concentrations are selected from at least two of the following: about 95% v/v, about 90% v/v, about 85% v/v, about 80% v/v, about 75% v/v, about 70% v/v, about 65% v/v, about 60% v/v, about 55% v/v, and about 50% v/v alcohol.

45. The method of claim 40 wherein the alcohol is ethanol.

46. The method of claim 1 having a reduced bio-burden level by at least one step in the method.

47. The method of claim 46 having a reduced bio-burden by including at least one antibiotic in the method.

48. The method of claim 47 wherein the antibiotic comprises penicillin, neomycin, amphotericin B. or streptomycin.

49. A tissue implant produced according to the method of claim 1.

50. A kit for preparing a tissue specimen, the kit comprising:
at least one detergent or instructions for preparing at least one detergent,
at least one nuclease or instructions for preparing at least one nuclease,
at least one hydrolase or instructions for preparing at least one hydrolase, and
instructions for treating a tissue specimen with the at least one detergent, nuclease and hydrolase to result in a substantially nonimmunogenic and substantially acellular tissue specimen.

51. The kit of claim 50 further comprising at least one tissue treatment reagent, the at least one tissue treatment reagent comprising:
at least one salt or instructions for preparing at least one salt,
at least one buffer or instructions for preparing at least one buffer,
at least three different alcohol concentrations or instructions for preparing at least three different alcohol concentrations, and
a wash solution or instructions for preparing a wash solution.

52. A method of implanting a tissue graft comprising disposing in an orthopedic tissue, a tissue graft treated by
lysing at least one cell of the tissue graft resulting in production of cellular debris,
enhancing a permeability of the extracellular matrix of the tissue graft,
degrading the cellular debris, and
removing the degraded cellular debris,
wherein any of the treatments may be repeated, and wherein the resulting tissue specimen is substantially acellular and substantially nonimmunogenic.
